(19) United States
(12) Patent Application Publication
(10) Pub. No.: US 2025/0161092 A1
(43) Pub. Date: May 22, 2025

(54) Strap System for Facilitating Cannulation in Obese Patients

(71) Applicant: CannuMate LLC

(72) Inventor: Alejandro Puglia, Beverly, MA (US)

(73) Assignee: Cannumate LLC, New York, NY (US)

(21) Appl. No.: 18/515,582

(22) Filed: Nov. 21, 2023

(51) Int. Cl. A61F 5/37

(52) U.S. Cl. CPC A61F 5/3776

(57) ABSTRACT

Disclosed is a strap system for moving adipose tissue in the abdominal region of a patient away from a cannulation site near the patient's groin. The strap system has a main strap of length to fit over a substantial portion of circumference of the abdominal skin surface, with a first adhesive suitable for bonding to the skin surface on one of the main strap surfaces, and attached to said main strap is at least one first transverse strap of length to fit over a substantial portion of the axial dimension of the ventral upper body skin surface and the axial dimension of the ventral upper body skin surface, and having said first adhesive on one of its surfaces. Attached to said main strap is at least one second transverse strap with a second adhesive on one of its surfaces of length to be secured to a portion of the bed frame or bed rails in a position to move abdominal adipose tissue away from the patient's groin. This facilitates cannulation in the groin area of obese patients.

### BACKGROUND

Central venous and arterial access are critical in intensive care, enabling a range of procedures including emergency treatments, hemodialysis, angiography, and arterial pressure monitoring. The femoral vein and artery are favored sites, but access can be problematic in obese patients, due to the overlay of abdominal adipose tissue. In obese patients, traditional access methods require several healthcare providers to manually retract this tissue, which is inefficient and complication-prone process.

Thus what is needed is a reliable mechanism to lift and hold a patient's abdominal adipose tissue for cannulation procedures.

### SUMMARY

The invention relates to an adhesive strap system for binding to a patient's abdominal skin surface and allowing the skin and underlying adipose tissue to be pulled away from the groin area, to facilitate cannulation or catheterization into veins in the groin area. In one embodiment, at least one main strap, with adhesive on one surface capable of bonding to the skin, is designed to be placed on the abdomen transverse to the longitudinal axis of the patient. More than one main strap may also be used.

The preferred adhesive component should have a strong bond to skin, for secure placement, but should also be readily removable, to minimize irritation or pain upon removal. Examples include the DermaBond Prineo 22™ system, and use of chlorine as a remover for it, as well as other commercially available skin adhesives, with solvents for removal.

Four other straps, with adhesive on one surface capable of bonding to the skin, are attached to the main strap(s) and extend substantially transversely to the main strap(s). These four transverse straps are designed to bind to the skin surface above (if the patient were standing) the area where the main strap(s) adheres. One or more of these transverse straps are designed to extend over the patient's shoulders to the upper part of the patients' back, and bind to the underlying skin surface from the main strap(s) to the upper part of the patients' back. One or more of these four transverse straps have sufficient length to attach to the hospital bed rails or to a portion of the bed frame near the patient's head. The adhesive can be used to bond to the rails or frame, or back to the strap itself, to fix the straps in place on or around the rails or frame.

### BRIEF DESCRIPTION OF THE DRAWINGS

FIG. 1 illustrates a patient with the device in place over the shoulders, and adhered to the dorsal and ventral skin surfaces.

### DETAILED DESCRIPTION

For cannulation to take place, first, a doctor anesthetizes the area with a local anesthetic. Then, a needle is placed into the blood vessel. A guide wire is put through the needle into the blood vessel and the needle is removed. The catheter is slid over the guide wire and moved into the blood vessel. The catheter is guided through the blood vessels until the tip is in the area to be studied.

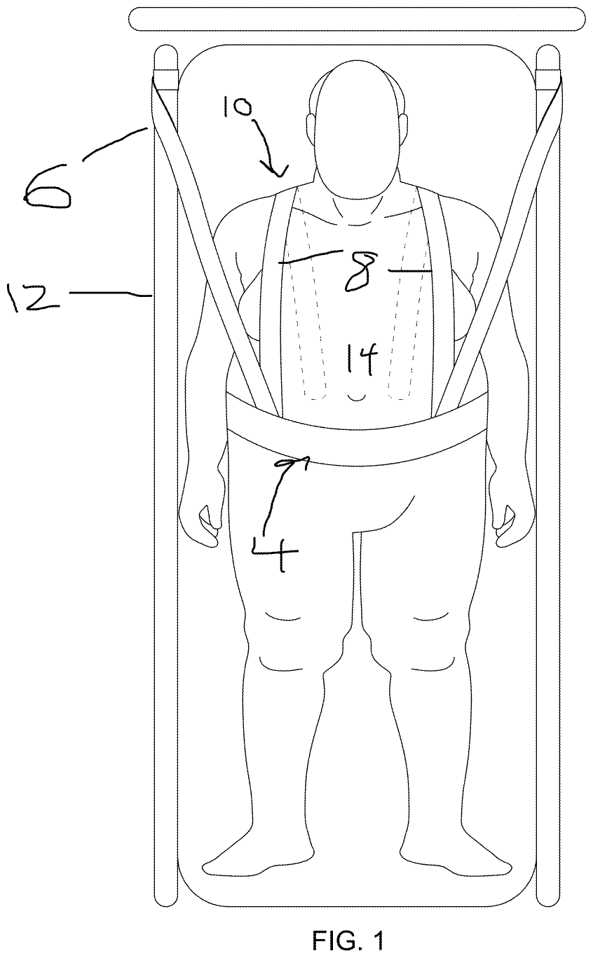

To facilitate access to veins in the groin area of obese patients, the adipose tissue of patient 14, which tends to obscure that area, can be pulled up with a strap system 10 as shown in FIG. 1. Strap system 10 has a main strap 4 directed along the circumference of the abdominal skin surface, and transverse straps 6 and 8, all with adhesive on one side. The straps 8 pass substantially axially over patient 14's shoulders and adhere along his ventral and dorsal upper body skin surface, as shown by the dotted lines in FIG. 1. Straps 6 are secured to bed rails or, as illustrated in FIG. 1, the bed frame 12. They can be wrapped around rails or the frame and adhered, as shown, to hold the adipose tissue in place before and during the cannulation procedure.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in embodiments or examples of the present invention, any of the terms “comprising” and “including” are to be read expansively and without limitation. The methods and processes illustratively described herein suitably may be practiced in differing orders of steps, and that they are not necessarily restricted to the orders of steps indicated herein or in the claims. It is also noted that as used herein and in the appended claims, the singular forms “a,” “an,” and “the” include plural reference, and the plural include singular forms, unless the context clearly dictates otherwise. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The invention has been described broadly and generically herein. Each of the narrower species and subgeneric groupings falling within the generic disclosure also form part of the invention. The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. A strap system for moving adipose tissue in the abdominal region of a patient away from a cannulation site near the patient's groin, comprising:
a main strap of length to fit over a substantial portion of circumference of the abdominal skin surface, with a first adhesive suitable for bonding to the skin surface on one of its surfaces, and attached to said main strap is at least one first transverse strap of length to fit over a substantial portion of the axial dimension of the ventral upper body skin surface and the axial dimension of the ventral upper body skin surface, and having said first adhesive on one of its surfaces, and
attached to said main strap is at least one second transverse strap with a second adhesive on one of its surfaces of length to be secured to a portion of the bed frame or bed rails in a position to move said adipose tissue away from the patient's groin.

2. The strap system of claim 1 wherein there are two first transverse straps.

3. The strap system of claim 1 wherein there are second transverse straps.

4. The strap of claim 3 wherein the second adhesive is different from said first adhesive.

5. The strap system of claim 1 wherein the first and second adhesives are the same type.

6. The strap system of claim 1 wherein the first adhesive can be readily removed from the skin surface using a solvent.

7. The strap system of claim 1 wherein the cannulation site is in the patient groin area.

8. The strap system of claim 1 wherein the first transverse strap is attached to the edge of the main strap.

9. The strap system of claim 1 wherein the second transverse strap is attached to the edge of the main strap.

10. A method of cannulation of an obese patient by moving adipose tissue in the abdominal region of a patient away from a cannulation site in the patient's groin area, comprising:
providing a strap system including: (a) a main strap of length to fit over a substantial portion of circumference of the abdominal skin surface, with a first adhesive suitable for bonding to the skin surface on one of its surfaces, and (b) in connection with said main strap is at least one first transverse strap of length to fit over a substantial portion of the axial dimension of the ventral upper body skin surface and the axial dimension of the ventral upper body skin surface, and having said first adhesive on one of its surfaces, and (c) in connection with said main strap is at least one second transverse strap with a second adhesive on one of its surfaces of length to be secured to a portion of the bed frame or bed rails in a position to move said adipose tissue away from the patient's groin;
adhering said main strap to a patient's ventral upper body skin surface;
adhering said first transverse strap to said patient's ventral upper body skin surface and over the patient's shoulders thereby also adhering to the patient's dorsal upper body skin surface; and
adhering the second transverse strap to a bed frame or to bed rails, such that said adipose tissue is pulled away from the cannulation site.

11. The method of claim 10 further including performing cannulation at said cannulation site.

12. The method of claim 10 further including providing and attaching an additional first transverse strap to said patient's ventral upper body skin surface and over the patient's shoulders, thereby also adhering to the patient's dorsal upper body skin surface.

13. The method of claim 10 further including providing and attaching at least one additional second transverse strap to the bed rails or the bed frame.

14. The method of claim 10 further including removing said first adhesive from the skin surface using a solvent.

15. The method of claim 10 wherein the connection of said first transverse strap with the main strap is direct attachment.

16. The method of claim 10 wherein the connection of said second transverse strap with the main strap is direct attachment.

17. The method of claim 10 wherein the main strap includes a commercially available tape.

18. The method of claim 10 wherein the first transverse strap includes a commercially available tape.

19. The method of claim 10 wherein the second transverse strap includes a commercially available tape.

20. The method of claim 10 wherein the first adhesive is removed with chlorine.
